(19) United States
(12) Patent Application Publication
(10) Pub. No.: US 2022/0249864 A1
(43) Pub. Date: Aug. 11, 2022

(54) METHOD BY WHICH BRAIN WITH ALZHEIMER'S DISEASE RECOVERS FROM BRAIN WAVE DAMAGE BY USING OPTOGENETICS

(71) Applicant: Korea University Research and Business Foundation, Seoul (KR)

(72) Inventors: Jeehyun KWAG, Seoul (KR); Kyerl PARK, Seoul (KR); Hyowon CHUNG, Busan (KR)

(73) Assignee: Korea University Research and Business Foundation, Seoul (KR)

(21) Appl. No.: 17/626,876

(22) PCT Filed: Nov. 25, 2020
(86) PCT No.: PCT/KR2020/016765

§ 371 Date: Jan. 13, 2022

(30) Foreign Application Priority Data
Dec. 26, 2019 (KR) 10-2019-0175301
Mar. 25, 2020 (KR) 10-2020-0036211

(51) Int. Cl. A61N 5/06; A61K 48/00; A01K 67/027

(52) U.S. Cl. CPC A61N 5/0622; A61K 48/0058; A61N 2005/063; A01K 2227/105; A01K 67/0278

(57) ABSTRACT

The present disclosure relates to a method for restoring brainwave impairment in Alzheimer's brain using optogenetic technology. The method for restoring brainwave impairment in Alzheimer's brain according to the present disclosure can restore brainwave impairment in Alzheimer's brain to a normal level by specifying inhibitory neurons associated with brainwave impairment in Alzheimer's disease and selectively activating impaired neurons through optical stimulation rather than electrical stimulation.

### TECHNICAL FIELD

The present disclosure relates to a method for restoring brainwave impairment in Alzheimer's brain using optogenetic technology, more particularly to a method for restoring brainwave impairment in Alzheimer's brain by selectively transmitting optical stimulation to parvalbumin (hereinafter, PV) or somatostatin (hereinafter, SST) in inhibitory neurons that play a critical role in generation of brainwaves through optogenetic technology without harming the neurons by using optical stimulation rather than electrical stimulation, thereby regulating the activity of the neurons and selectively restoring theta wave and gamma wave impairment in Alzheimer's brain.

### BACKGROUND ART

Alzheimer's disease is a representative degenerative brain disease. It is known that abnormal amounts of amyloid beta accumulating in the brain cause impairment of learning and memory.

In normal brain, brainwaves of various frequency bands, including delta wave (0.1-3 Hz), theta wave (3-12 Hz), alpha wave (8-15 Hz), beta wave (16-30 Hz) and gamma wave (20-120 Hz), are generated for memory formation due to inhibition of various inhibitory neurons. The generation of brainwaves is inhibited in Alzheimer's brain due to decline in the function of the inhibitory neurons caused by accumulation of amyloid beta, which may cause decline in learning and memory occurring in Alzheimer's disease.

A method of restoring brainwave impairment in Alzheimer's brain by inserting an electrode and providing high-frequency-band electrical stimulation has been presented.

However, it has the problem that neurons may be damaged and selective regulation of the various functions and activities of inhibitory neuron in the brain cannot be achieved with the non-selective electrical stimulation.

Therefore, a technology of treating neurological disorders through optogenetics rather than electrical stimulation has been proposed. For example, patent document 1 discloses a method for optical stimulation therapy including: a step of transfecting a target tissue with a light-sensitive channel protein sensitive to light in a specific wavelength range; a step of delivering light in the wavelength range to the target tissue via an optical stimulation device; a step of sensing bioelectric signals substantially simultaneously with the delivering of the light to the table tissue; a step of determining a patient's therapeutic state based on the bioelectric signals; and a step of adjusting the delivery of the light to the target tissue based on the sensed patient's therapeutic state, and describes that the method can be applied to neurological disorders such as depression, dementia, Parkinson's disease, spasticity, epilepsy, etc.

In addition, patent document 2 discloses a method for treating depression through optogenetic stimulation of the left medial prefrontal cortex.

Specifically, it is described that stress resulting from social interaction is closely related with the left medial prefrontal cortex and the stress can be regulated through optical stimulation of the left medial prefrontal cortex, which expresses channelrhodopsins in the left medial prefrontal cortex and is useful for treatment of depression by providing information necessary for the regulation of the stress.

Although the possibility of the optogenetic technology for treatment of neurological disorders has been proposed, the treatment of Alzheimer's disease using optogenetic technology has not been reported yet. In particular, there is no technology capable of selectively restoring brainwaves in a specific wavelength range.

The inventors of the present disclosure have made efforts to solve the problems of the existing art. As a result, they have completed the present disclosure by providing a technology capable of selectively regulating the activity of PV and SST inhibitory neurons, which are known to play a critical role in generation of brainwaves, without harming the neurons by using optical stimulation rather than electrical stimulation. They have identified that, by regulating the activity of the PV and SST inhibitory neurons by expressing photoreceptors only in the inhibitory neurons, theta wave impairment in Alzheimer's brain can be restored through optogenetic activation of the SST inhibitory neurons and gamma wave impairment in Alzheimer's brain can be restored through optogenetic activation of the PV inhibitory neurons.

### REFERENCES OF RELATED ART

### Patent Documents

US Patent Publication No. 2011-0125078 (published on May 26, 2011). Korean Patent Publication No. 2014-0125659 (published on Oct. 29, 2014).

### DISCLOSURE

### Technical Problem

The present disclosure is directed to providing a method for restoring brainwave impairment in Alzheimer's brain using optogenetic technology.

### Technical Solution

The present disclosure provides a method for restoring brainwave impairment in Alzheimer's brain using optogenetic technology, which includes:

a step of preparing an animal model in which the CRE gene is expressed in inhibitory neurons;

a step of injecting a viral vector including a photoreceptor gene designed to target inhibitory neurons wherein the CRE gene is expressed into the animal model and expressing the viral vector;

a step of inserting an electrode and an optical fiber into the animal model into which the viral vector has been injected; and

a step of restoring brainwave impairment in Alzheimer's brain by transmitting optical stimulation to the designed photoreceptor gene to be selectively expressed only in the target neurons through the electrode and the optical fiber.

In the present disclosure, a step of quantifying the restoration of brainwave impairment may be performed after the step of restoring brainwave impairment in Alzheimer's brain.

The inhibitory neurons may be neurons expressing PV or SST, and gamma wave impairment or theta wave impairment may be restored selectively by the optical stimulation.

In the present disclosure, the photoreceptor may be a photoreceptor that responds to light with a wavelength of 450, 470, 565 or 590 nm.

### Advantageous Effects

A method for restoring brainwave impairment in Alzheimer's brain using optogenetic technology of the present disclosure can restore brainwave impairment in Alzheimer's brain by regulating the activity of neurons using optical stimulation without damaging the neurons unlike the existing method of using electrical stimulation.

In addition, unlike the existing method of applying electrical stimulation to an unspecified number of neurons non-selectively, the method for restoring brainwave impairment in Alzheimer's brain using optogenetic technology of the present disclosure allows selective regulation of only one type of cells among various inhibitory neurons of the brain using the genetic manipulation technology of inserting a specific photoreceptor gene using a viral vector.

In addition, the present disclosure allows the treatment of brainwave impairment in Alzheimer's brain by mimicking the brainwave generation mechanism of normal brain because it can induce neuronal activation pattern similar to the activation pattern of inhibitory neurons occurring during generation of brainwaves in normal brain using various optical stimulation patterns in Alzheimer's brain.

### BRIEF DESCRIPTION OF DRAWINGS

FIG. 1 illustrates a process of Aβ oligomer synthesis.

FIG. 2 shows illustrates optogenetic manipulation technique and injection of a viral vector and Aβ using sterotactic technique.

FIG. 3 shows Aβ expression and SST/PV neurons in the hippocampus of mouse with Alzheimer's disease.

FIG. 4 shows restoration of theta wave impairment through optogenetic regulation of the activity of SST neurons in the hippocampus of mouse with Alzheimer's disease.

FIG. 5 shows restoration of gamma wave impairment through optogenetic regulation of the activity of PV neurons in the hippocampus of mouse with Alzheimer's disease.

### BEST MODE

Hereinafter, the present disclosure is described in detail.

The present disclosure provides a method for restoring brainwave impairment in Alzheimer's disease by inducing neuronal activation pattern similar to the activation pattern of inhibitory neurons occurring during in normal brain using various optical stimulation patterns in Alzheimer's brain, thereby mimicking the brainwave generation mechanism of normal brain.

Specifically, the present disclosure provides a method for restoring brainwave impairment in Alzheimer's brain using optogenetic technology, which includes:

1) a step of preparing an animal model in which the CRE gene is expressed in inhibitory neurons;

2) a step of injecting a viral vector including a photoreceptor gene designed to target inhibitory neurons wherein the CRE gene is expressed into the animal model and expressing the viral vector;

3) a step of inserting an electrode and an optical fiber into the animal model into which the viral vector has been injected; and

4) a step of restoring brainwave impairment in Alzheimer's brain by transmitting optical stimulation to the designed photoreceptor gene to be selectively expressed only in the target neurons through the electrode and the optical fiber.

Hereinafter, each step is described in detail referring to the attached drawings.

In the method for restoring brainwave impairment in Alzheimer's brain using optogenetic technology of the present disclosure, the step 1) is a step of preparing an animal model of Alzheimer's disease. The animal model of Alzheimer's disease is prepared by inducing Alzheimer's disease by injecting Aβ oligomers, which are soluble and have the strongest toxicity among Aβ peptides, directly into the brain, or an animal model of Alzheimer's disease that has been established through the existing genetic manipulation technology is used.

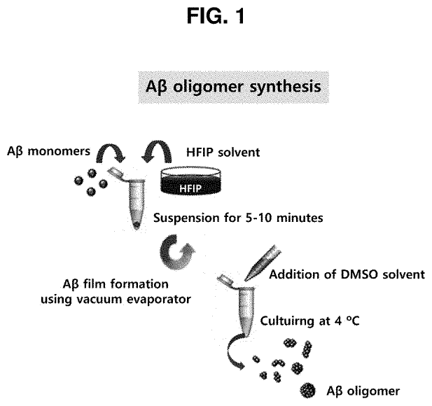

FIG. 1 illustrates a process of Aβ oligomer synthesis. After suspending Aβ monomers in HFIP (hexafluoro-2-propanol) as an organic solvent for 5-10 minutes, a film is formed by removing HFIP from the Aβ suspension using a vacuum evaporator. An Aβ suspension is prepared by treating the Aβ film with DMSO (dimethyl sulfoxide) while slowing the aggregation of Aβ. The Aβ suspension is diluted to 200 nM by treating with artificial cerebrospinal fluid and then soluble Aβ oligomers are synthesized by incubating in a refrigerator for about 12 hours. The synthesized Aβ oligomers are injected directly into the brain of an animal by a stererotactic method to induce Alzheimer's disease.

The previously established animal models of Alzheimer's disease of the step 1) include Alzheimer's disease animal models established through genetic manipulation such as 5×FAD mice, APP/PS1 mice, etc.

In the step 1), PV or SST neurons are used as the inhibitory neurons and the CRE gene is expressed in the specific inhibitory neurons.

The CRE refers to CRE recombinase, which recognizes, cleaves and ligates a DNA of a specific sequence. For example, a PV-CRE animal model is an animal model having the CRE gene. A PV-expressing promoter is located upstream of the CRE gene and, thus, the CRE enzyme is expressed only in the PV-expressing neurons.

In the method of the present disclosure, the step 2) is a step of preparing a viral vector including a photoreceptor gene designed to target inhibitory neurons wherein the CRE gene is expressed and injecting the viral vector including the photoreceptor gene into the brain of the CRE transgenic animal model of the step 1).

The viral vector refers to an adeno-associated virus having a promoter such as EF1a-DIO, CAG-Flex and Syn-Flex. It can deliver the photoreceptor gene by targeting only the neurons that express the CRE gene.

The photoreceptor includes all types of photoreceptors that increase the activity of the neurons expressing photoreceptors in response to light. Specific examples include ChIEF which responds best to the light with a wavelength of 450 nm, channelrhodopsin-2 (hereinafter, ChR2) which responds best to the light with a wavelength of 470 nm, C1V1 which responds best to the light with a wavelength of 565 nm, Crimson which responds best to the light with a wavelength of 590 nm, etc. In addition, all types of photoreceptors that can activate neurons can be used as the photoreceptor of the step 2).

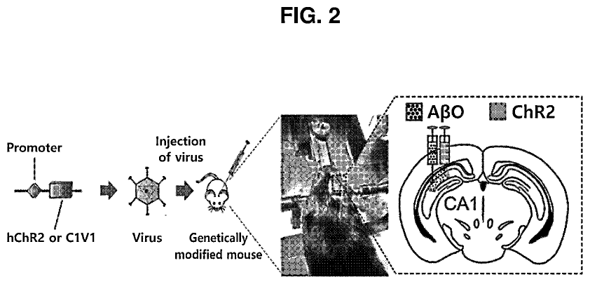

FIG. 2 shows illustrates the optogenetic manipulation technique and injection of the viral vector and Aβ using sterotactic technique. Into the brain of the CRE transgenic animal model, more specifically an SST-CRE or PV-CRE animal model, the viral vector encoding the photoreceptor may be injected using the stererotactic method, such that the photoreceptoris expressed selectively only in SST and PV neurons.

After the viral vector is injected, the viral vector is expressed in the specific inhibitory neurons including the CRE gene for 2-3 weeks for genetic modification.

In the method of the present disclosure, the step 3) is a step in which an electrode and an optical fiber are inserted the animal model into which the viral vector has been injected. An electrode and an optical fiber are inserted for brainwave measurement and delivery of optical stimulation in the brain of the Alzheimer's disease animal model.

Specifically, for in-vitro situation, brainwaves may be measured by inserting a glass microelectrode into the brain of the Alzheimer's disease animal model and may be transmitted to a photoelectrode using a light source and a microscopic lens.

And, for in-vivo situation, after drilling the skull of the anesthetized animal and determining the site of the brain into which the electrode will be inserted using the sterotactic method, the photoelectrode (electrode+optical fiber) may be injected and then the surgical site may be covered with dental cement.

Then, in the step 4) of the present disclosure, the brainwave impairment in Alzheimer's brain is restored selectively by delivering optical stimulation using the electrode and the optical fiber.

In the step 4), the Alzheimer's brain is optically stimulated by irradiating light with a wavelength of 450, 470, 565 or 590 nm. Through the optical stimulation, the activity of specific inhibitory neurons wherein photoreceptors other than ChIEF, ChR2, C1V1 and Crimson are expressed is regulated.

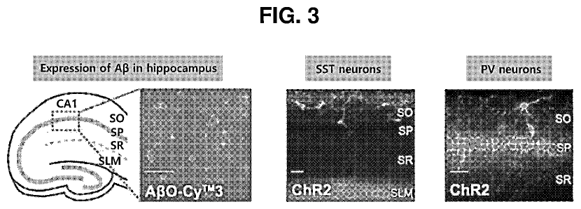

FIG. 3 shows Aβ expression and SST/PV neurons in the hippocampus of mouse with Alzheimer's disease. The ChR2-expressing SST/PV neurons in the Alzheimer's brain can be selectively activated through optical stimulation at 470 nm of the SST-CRE or PV-CRE mouse hippocampus into which Aβ and the virus have been injected. Through this, theta wave impairment and gamma wave impairment may be restored by transmitting optical stimulation to the Alzheimer's brain.

The optical stimulation becomes different depending on the photoreceptor. Light with a wavelength of 470 nm is transmitted for ChR2, and light with a wavelength of 565 nm is transmitted for C1V1 using the photoelectrode inserted in the brain.

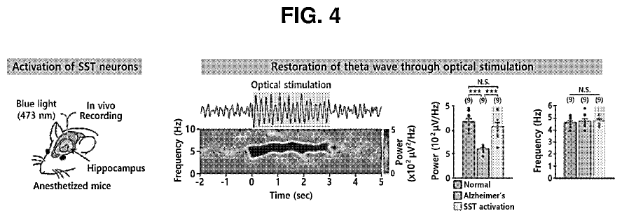

FIG. 4 shows restoration of theta wave impairment through optogenetic regulation of the activity of SST neurons in the hippocampus of mouse with Alzheimer's disease. The restoration of the theta wave impairment is achieved by activating the SST neurons in which the photoreceptor is expressed through optical stimulation.

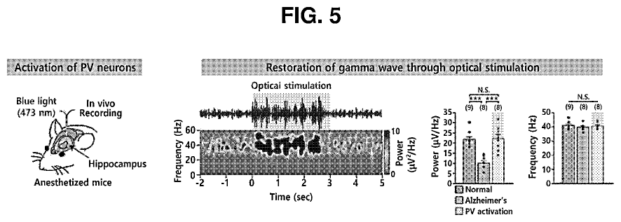

FIG. 5 shows restoration of gamma wave impairment through optogenetic regulation of the activity of PV neurons in the hippocampus of mouse with Alzheimer's disease. The restoration of the impaired gamma wave is achieved by activating the PV neurons in which the photoreceptor is expressed through optical stimulation.

The intensity and pattern of optical stimulation are provided to mimic the various activation patterns occurring in normal brain depending on the type of inhibitory neurons. For example, the activation pattern of parvalbumin neurons in the gamma wave range of normal brain may be mimicked by stimulating the ChR2-expressing parvalbumin neurons of Alzheimer's brain with a sine wave pattern of light with a wavelength of 470 nm and a frequency band of 5 Hz to restore the gamma wave impairment of the Alzheimer's disease. It is to be understood that the intensity of the optical stimulation may be proportional to the degree of brainwave impairment occurring in the Alzheimer's brain.

In addition, the pattern of the optical stimulation may be a continuous stimulation pattern, a sine wave pattern, a specific frequency pattern, etc.

The method for restoring brainwave impairment in Alzheimer's brain using optogenetic technology of the present disclosure may further include, after the step 4) of restoring the brainwave impairment in Alzheimer's brain, 5) a step of quantifying the restoration of the brainwave impairment by verifying the restoration of the brainwave impairment.

The restoration of the brainwave impairment in the Alzheimer's brain may be quantified by band-pass filtering brainwaves and analyzing power through fast Fourier transform, etc.

The method for restoring brainwave impairment in Alzheimer's brain using optogenetic technology allows restoration of brainwave impairment in Alzheimer's brain by regulating the activity of neurons using optical stimulation without harming the neurons.

In addition, the method for restoring brainwave impairment in Alzheimer's brain using optogenetic technology of the present disclosure allows selective activation of only one type of inhibitory neurons from among various inhibitory neurons through genetic manipulation of inserting a photoreceptor gene into specific neurons, unlike the existing method of non-selectively applying electrical stimulation to an unspecified number of neurons.

In particular, the present disclosure allows restoration of theta wave and gamma wave impairment which are known to be closely related to memory impairment in Alzheimer's brain.

Although the specific exemplary embodiments of the present disclosure have been described in detail above, it will be obvious to those skilled in the art that various changes and modifications can be made within the scope of the present disclosure and such changes and modifications are included in the scope of the appended claims.

Hereinafter, the present disclosure will be described in more detail through examples.

The following examples are provided to illustrate the present disclosure more specifically and the scope of the present disclosure is not limited by the examples.

### <Example 1> Restoration of Theta Wave Impairment in Alzheimer's Brain

1. Establishment of Alzheimer's Disease Animal Model Through Injection of Amyloid Beta Oligomers

Aβ monomers were suspended in an HFIP (hexafluoro-2-propanol) solvent at a concentration of 1 mM and then a film was formed by removing HFIP from the Aβ suspension using a vacuum evaporator. A 5 mM Aβ suspension was prepared by treating the Aβ film with a DMSO solvent while slowing the aggregation of Aβ. The Aβ suspension was diluted to 200 nM by treating with physiological saline and then soluble Aβ oligomers were synthesized by incubating in a refrigerator (4° C.) for about 12 hours.

3 μL of the synthesized Aβ oligomers were injected directly into the hippocampus CA1 of SST-CRE mice by a stererotactic method. The Aβ oligomers were injected at a speed of 100 nL/min using a microsyringe, and the microsyringe was held tight for 3 minutes so that the Aβ oligomers could spread well.

2. Injection of Virus for Optogenetic Technology

For expression of a photoreceptor in SST neurons, ChR2 was used as a photoreceptor. The AAV-EF1a-DIO-C1V1 (E162T)-TS-p2A-EYFP-WPRE virus (1 μL) was injected into the hippocampus CA1 of the SST-CRE mice into which the β oligomers were injected by a stererotactic method.

The virus was injected at a speed of 100 nL/min using a microsyringe, and the microsyringe was held tight for 3 minutes so that the virus could spread well. Then, the mice were allowed to recover for 3 weeks.

3. Insertion of Electrode and Optical Fiber into Animal Model into with Animal Model with Viral Vector Injected

For measurement of brainwaves from the hippocampus CA1 of the mice of the Alzheimer's model, a photoelectrode was inserted into the brain of the anesthetized mice by a stererotactic method.

The photoelectrode included an electrode for measurement of brainwaves and an optical fiber for transmission of optical stimulation. Theta wave was obtained by band-pass filtering with a local field potential in a frequency band of 3-12 Hz using the electrode, and the power value and frequency band of the theta wave were obtained through fast Fourier transform.

4. Verification of Restoration of Theta Wave Impairment in Alzheimer's Disease Animal Model Hippocampus Through Optical Stimulation

For recover of theta wave impairment, light with a wavelength of 470 nm was irradiated to the hippocampus of the SST-CRE mice into which the Aβ oligomers and virus were injected using the photoelectrode. The optical stimulation was transmitted continuously with a constant intensity for 3 seconds. As a result, it was confirmed that the theta wave impairment in the hippocampus of the mice of the Alzheimer's disease model was recovered to a normal level when the SST inhibitory neurons were activated through optogenetic optical stimulation [FIG. 4].

### <Example 2> Restoration of Gamma Wave Impairment in Alzheimer's Brain

After synthesizing amyloid beta (Aβ) oligomers in the same manner as in Example 1, 3 μL of the synthesized Aβ oligomers were injected into the hippocampus CA1 of PV-CRE mice by a stererotactic method. The Aβ oligomers were injected at a speed of 100 nL/min using a microsyringe, and the microsyringe was held tight for 3 minutes so that the Aβ oligomers could spread well.

For expression of a photoreceptor in PV neurons, ChR2 was used as a photoreceptor. The AAV-EF1a-DIO-C1V1(E162T)-TS-p2A-EYFP-WPRE virus (1 μL) was injected into the hippocampus CA1 of the PV-CRE mice into which the β oligomers were injected by a stererotactic method. The virus was injected at a speed of 100 nL/min using a microsyringe, and the microsyringe was held tight for 3 minutes so that the virus could spread well. Then, the mice were allowed to recover for 3 weeks.

For measurement of brainwaves from the hippocampus CA1 of the mice of the Alzheimer's model, a photoelectrode was inserted into the brain of the anesthetized mice by a stererotactic method. Gamma wave was obtained by band-pass filtering with a local field potential in a frequency band of 20-120 Hz using the electrode, and the power value and frequency band of the gamma wave were obtained through fast Fourier transform.

For recover of gamma wave impairment, light with a wavelength of 470 nm was irradiated to the hippocampus of the PV-CRE mice into which the Aβ oligomers and virus were injected using the photoelectrode. The optical stimulation was transmitted continuously with a constant intensity for 3 seconds. As a result, it was confirmed that the gamma wave impairment in the hippocampus of the mice of the Alzheimer's disease model was recovered to a normal level when the PV inhibitory neurons were activated through optogenetic optical stimulation [FIG. 5].

Although the specific exemplary embodiments of the present disclosure have been described in detail above, it will be obvious to those skilled in the art that various changes and modifications can be made within the scope of the present disclosure and such changes and modifications are included in the scope of the appended claims.

1. A method for restoring brainwave impairment in Alzheimer's brain using optogenetic technology, comprising:
a step of preparing an animal model in which the CRE gene is expressed in inhibitory neurons;
a step of injecting a viral vector comprising a photoreceptor gene designed to target inhibitory neurons wherein the CRE gene is expressed into the animal model and expressing the viral vector;
a step of inserting an electrode and an optical fiber into the animal model into which the viral vector has been injected; and
a step of restoring brainwave impairment in Alzheimer's brain by transmitting optical stimulation to the designed photoreceptor gene to be selectively expressed only in the target neurons through the electrode and the optical fiber.

2. The method for restoring brainwave impairment in Alzheimer's brain using optogenetic technology according to claim 1, wherein a step of quantifying the restoration of brainwave impairment is performed after the step of restoring brainwave impairment in Alzheimer's brain.

3. The method for restoring brainwave impairment in Alzheimer's brain using optogenetic technology according to claim 1, wherein the inhibitory neurons are neurons expressing parvalbumin or somatostatin.

4. The method for restoring brainwave impairment in Alzheimer's brain using optogenetic technology according to claim 1, wherein the photoreceptor is a photoreceptor that responds to light with a wavelength of 450, 470, 565 or 590 nm.

5. The method for restoring brainwave impairment in Alzheimer's brain using optogenetic technology according to claim 1, wherein gamma wave impairment or theta wave impairment is restored selectively by the optical stimulation.
